(19) United States
(12) Patent Application Publication
(10) Pub. No.: US 2012/0323158 A1
(43) Pub. Date: Dec. 20, 2012

(54) EXTRACORPOREAL IMMUNOADSORPTION TREATMENT

(76) Inventor: Paul Tebbey, Gurnee, IL (US)

(21) Appl. No.: 13/580,352

(22) PCT Filed: Mar. 10, 2011
(86) PCT No.: PCT/US11/27997

§ 371 Date: Aug. 21, 2012

Related U.S. Application Data
(60) Provisional application No. 61/312,558, filed on Mar. 10, 2010.

(51) Int. Cl. A61M 1/14

(52) U.S. Cl. 604/6.01; 604/5.01

(57) ABSTRACT

A method for extracorporeally administering selected biologic agents that target soluble cytokines and chemokines via immunoapheresis in order to treat patients with a variety of acute or chronic autoimmune and inflammatory disease states.

### FIELD OF THE INVENTION

The present invention generally relates to the fields of immunology and immune-mediated inflammatory diseases, in particular to an extracorporeal treatment targeting soluble cytokines, chemokines or other growth modulators in blood plasma or interstitial cellular fluids.

### BACKGROUND

Biologic therapies are generally injected into the body of a subject, such as through intravenous (IV), intra-muscular (IM) or sub-cutaneous (SC) injection, in order to effect treatment. There are however disadvantages to this treatment modality. For example, the biologic agent may remain in a subject's body for an extended period of time even after the targeted agent (typically a cytokine, chemokine or other growth factor) has already been removed and is no longer stimulating an inflammatory response. This over-suppression potentially exposes the patient to undesirable rare infections that in some cases are life-threatening. Chronic administration of a biological agent in this manner can also result in injection site reactions and infusion reactions which impede the agent's effectiveness in the long term due to host immune response to the injected therapeutic agent.

### SUMMARY

The present invention solves the foregoing problems by adsorbing one or more biologic agents to a column external to the body and passing blood or plasma through the column containing the biologic agent or agents. One or more targeted inflammatory cytokines, chemokines, and/or growth factors are removed from the blood or plasma by the biologic agents, resulting in reduced levels of such cytokines, chemokines, and/or growth factors in the blood or plasma, which is then returned to the subject's circulation.

The present system for treating an autoimmune or inflammatory disease of a subject comprises a vascular access conduit adapted at a first end for removing a body fluid from the subject, such as blood or plasma containing a cytokine or a growth factor. The vascular access conduit can be directly connected to the hub of a needle inserted into the patient's vasculature or to another direct connection to the subject's vasculature, such as an implanted access port. Alternatively, the access conduit can be connected to another source of the subject's body fluids, such as an outlet of a plasma separation device or apheresis unit, as shown in FIG. 2. Such an apheresis unit can comprise a centrifuge for removing blood cells and other blood components that might tend to clog the chromatography unit of the present system.

The present system further includes a chromatography system comprising a solid substrate that retains one or more specific binding partners for a cytokine or a growth factor. The inlet end of the chromatography system is in fluid communication with a second end of the vascular access conduit, while the outlet end of the chromatography system is in fluid communication with a fluid return conduit which returns the processed blood plasma or other body fluid of the subject to the subject's vasculature.

The specific binding partners of the chromatography system are preferably antibodies, such as monoclonal antibodies, although any of a number of such binding partners can be used in the present system. For example, the specific binding partners can be complete antibodies, antibody fusion proteins, antibody fragments such as single-chain Fv fragments (scFv) or Fab fragments, diabodies, minibodies, single antibody domains and the like. Preferably, the antibodies are monoclonal antibodies, such as such commercially available monoclonal antibodies as tocilizumab, etanercept, adalimumab, infliximab, golimumab and ustekinumab. The antibodies or other specific binding partners specifically bind cytokines or growth factors such as an interferon, a tumor necrosis factor (TNF), an interleukin (IL), or a chemokine. Preferably, the cytokine or growth factor is one of the following molecules: interferon alpha, interferon beta, interferon gamma, TNF-alpha, TNF-beta, IL-1, IL-2, IL-3, IL-4, IL-5, IL-6, IL-7, IL-8, IL-10, IL-12, IL-13, IL-15, IL-17, IL-18, IL-20, IL-22, IL-23, MCP-1, MIF, RANTES, MIP-1-alpha, VEGF, FGF, EGF, or PDGF.

Such antibodies or other specific binding partners are immobilized in or otherwise retained by the solid substrate of the chromatography system, which can for example be a chromatography column having a solid substrate consisting of sepharose, agarose, and/or silica gel. In one embodiment, the substrate comprises a polymer matrix that excludes blood cells.

In another aspect, the present invention relates to the use of such a chromatography system to treat an autoimmune or inflammatory disease of a subject. In this embodiment, a body fluid such as blood comprising a cytokine and/or a growth factor is removed from the subject, and the body fluid or a fraction thereof, such as plasma, is conducted through the solid substrate of the chromatography system, where it is placed in contact with the specific binding partners. The body fluid or fraction thereof is then returned to the patient after it is placed in contact with the specific binding partners. The disease being treated in this embodiment can, for example, be rheumatoid arthritis, Crohn's disease, psoriasis, age-related macular degeneration, Alzheimer's disease, asthma, COPD, graft-versus-host disease, pulmonary eosinophilia, multiple sclerosis, systemic lupus erythematosus, sepsis, or cancer.

In a further aspect, the present invention relates to a method for treating an autoimmune or inflammatory disease of a subject. This method comprises the step of removing a body fluid from the subject, generally blood or plasma, which comprises a cytokine and/or a growth factor. The body fluid or a fraction thereof is then conducted to a substrate having one or more specific binding partners for the cytokine or the growth factor bound thereto, and the body fluid or fraction thereof is placed in contact with the specific binding partners, which bind and retain at least some of the targeted cytokine or a growth factor. After such treatment, the amount of the targeted cytokine or growth factor in the body fluid or fraction is reduced, and the body fluid or fraction thereof is then returned to the patient.

### FIGURES

FIG. 1 is a diagram illustrating the centrifugation of blood into different components.

FIG. 2 is a diagram illustrating an apheresis and immunoadsorption apparatus according to the present invention.

FIG. 3 is a diagram of an ambulatory apparatus of the present invention.

### DETAILED DESCRIPTION OF THE INVENTION

The present system and treatment obviate many of the deleterious effects of injection therapy described above and facilitate the chronic use of biologic agents, thereby resulting in better patient treatment outcomes. By virtue of not having an immunosuppressive biologic agent constantly in the body, the body's immune system is permitted to react normally to infectious agents or other immune perturbations such as tumorigenic processes.

Patients can be immunoapheresed with the present system every few months to remove abnormally high levels of cytokines or growth factors that have built up in the subject, for example as a result of the use of a biologic agent such as ACTEMRA (tocilizumab), ENBREL (etanercept), HUMIRA (adalimumab), REMICADE (infliximab), SIMPONI (golimumab) and STELARA (ustekinumab), while returning necessary plasma and blood components to the body. In one embodiment, an external device having filtration capabilities and a pump is used, wherein blood is immunoapheresed for extended periods to normalize the levels of disease-causing cytokines. Exact administration profiles can be optimized to the individual biologic agent or agents. The present invention can support longer term use of biologic agents, and can also permit the simultaneous use of a combination of biologic agents, since the negative consequences of combination therapy are avoided through the use of the present extracorporeal system.

### DEFINITIONS

As used herein, the following terms and variations thereof have the meanings given below, unless a different meaning is clearly intended by the context in which such term is used.

“Chemokine” refers to a type of cytokine, generally a small protein molecule, that activates immune cells, stimulates their migration, and helps direct immune cell traffic throughout the body.

“Chromatography” refers to a process for separating materials using two phases, one stationary and one moving, in particular the separation of components from a liquid by passing the liquid through a porous solid which binds one or more components in the liquid.

“Chromatography cartridge” refers to a housing containing the solid components of a chromatography system through which a liquid material passes.

“Cytokine” refers to any of a number of substances, (including lymphokines, interleukins and chemokines) that are secreted by specific cells of the immune system which carry signals locally between cells, and thus have an effect on other cells. They are a category of signaling molecules that are used extensively in cellular communication. Cytokines occur as proteins, peptides, and glycoproteins.

“Fractionate” refers to the separation of a mixture into different component portions by exploiting differences in a chemical or physical property, such as particle size or solubility. For example, blood can be fractionated into plasma and one or more cellular components by centrifugation. A “fraction” is a component portion of a mixture, e.g. plasma is a fraction of blood.

“Growth factor” refers to a substance that stimulates cell differentiation and growth, typically a protein or steroid.

“Specifically binds” refers to a binding reaction between a binding molecule and a target ligand in a heterogeneous population of molecules, such as plasma. Under designated conditions (e.g., immunoassay conditions in the case of an immunoglobulin), the binding molecule binds to its particular target and does not bind in a significant amount to other molecules present in a sample. “Specific binding partner” refers to molecule, such as a monoclonal antibody, that binds a target, such as a cytokine, chemokine, or growth factor.

The terms “patient,” “subject” and the like with reference to individuals that can be treated with the present methods refer to humans and other mammals.

The term “comprise” and variations of the term, such as “comprising” and “comprises,” are not intended to exclude other additives, components, integers or steps. The terms “a,” “an,” and “the” and similar referents used in the context of describing the present invention are to be construed to cover both the singular and the plural, unless otherwise indicated herein or clearly contradicted by context.

### Cytokines and Chemokines

The term cytokine encompasses a large and diverse family of polypeptide regulators that are produced widely throughout the body by cells of diverse embryological origin. The term “cytokine” has been used to refer to immunomodulating agents (interleukins, interferons, etc.). While classic protein hormones circulate in nanomolar (10⁻⁹) concentrations that usually vary by less than one order of magnitude, some cytokines (such as IL-6) circulate in picomolar (10⁻¹²) concentrations that can increase up to 1,000-fold during trauma or infection. The widespread distribution of cellular sources for cytokines can also differentiate them from hormones. Virtually all nucleated cells, but especially endo/epithelial cells and resident macrophages (many near the interface with the external environment), are potent producers of IL-1, IL-6, and TNF-α. In contrast, classic hormones, such as insulin, are secreted from discrete glands (e.g., the pancreas).

Each cytokine also has a matching cell-surface receptor. Subsequent cascades of intracellular signaling then alter cell functions. This can include the upregulation and/or downregulation of several genes and their transcription factors, resulting in the production of other cytokines, an increase in the number of surface receptors for other molecules, or the suppression of their own effect by feedback inhibition.

The term chemokine refers to a family of small cytokines, or proteins secreted by cells. The name is derived from their ability to induce directed chemotaxis in nearby responsive cells; they are chemotactic cytokines. Proteins are classified as chemokines according to shared structural characteristics such as small size (they are all approximately 8-10 kilodaltons in size), and the presence of four cysteine residues in conserved locations that are key to forming their 3-dimensional shape. However, these proteins have historically been known under several other names including the SIS family of cytokines, SIG family of cytokines, SCY family of cytokines, Platelet factor-4 superfamily or intercrines. Some chemokines are considered pro-inflammatory and can be induced during an immune response to promote cells of the immune system to a site of infection, while others are considered homeostatic and are involved in controlling the migration of cells during normal processes of tissue maintenance or development. These proteins exert their biological effects by interacting with G protein-linked transmembrane receptors called chemokine receptors, which are selectively found on the surfaces of their target cells.

Cytokines can be grouped through structural homology into the following groups, each of which falls under the scope of the present invention:
1. The four α-helix bundle family. Member cytokines have three-dimensional structures with four bundles of a-helices. This family in turn is divided into three sub-families:
   a. IL-2 subfamily
   b. interferon (IFN) subfamily
   c. IL-10 subfamily.
The first of these three subfamilies is the largest. It contains several non-immunological cytokines including erythropoietin (EPO) and thrombopoietin (THPO). Also, four α-helix bundle cytokines can be grouped into long-chain and short-chain cytokines.
2. The IL-1 family, which primarily includes IL-1 and IL-18
3. The IL-17 family, which has yet to be completely characterized, though member cytokines have a specific effect in promoting proliferation of T-cells that cause cytotoxic effects.

Immunological cytokines can also be divided into those that enhance cytokine responses, type 1 (IFN-γ, TGF-β, etc.), and type 2 (IL-4, IL-10, IL-13), which favor antibody responses. A key focus of interest has been that cytokines in one of these two subsets tend to inhibit the effects of those in the other. Dysregulation of this tendency is under intensive study for its possible role in the pathogenesis of autoimmune disorders. Several inflammatory cytokines are induced by oxidant stress. The fact that cytokines themselves trigger the release of other cytokines and lead also to increased oxidant stress makes them important in chronic inflammation and related immune mediated disorders.

The present system can be used to remove the following cytokines from a subject in order to effect treatment interferons (IFN-alpha, beta and gamma), tumor necrosis factors (TNF-alpha, TNF-beta), interleukins (IL-1, IL-2, IL-3, IL-4, IL-5, IL-6, IL-7, IL-8, IL-10, IL-12, IL-13, IL-15, IL-17, IL-18, IL-20, IL-22, IL-23), chemokines (MCP-1, MIF, RANTES, MIP-1-alpha), and growth factors (VEGF, EGF, PDGF).

### Inflammatory and Autoimmune Diseases

Autoimmune diseases have an overall prevalence of about 3% of the world population and have an incidence that is influenced by genetics, gender, and the environment. It is currently thought that the immune response of a genetically predisposed individual to an environmental pathogen, under the influence of inadequate or non-functional immunoregulatory mechanisms, can lead to the development of an autoimmune disease. Advances in the treatment of autoimmune diseases follow a better understanding of the abnormalities in the cellular activity pathways and the resulting, often permanent, imbalance of the pro- and anti-inflammatory cytokine expression profiles. Over the past few years, there has been a dramatic change in the therapeutic regimens employed in autoimmune diseases, with soluble receptors, monoclonal antibodies and molecular mimetics enhancing or gradually replacing conventional immunosuppressive therapies. New biologic agents (primarily proteins) have been developed, targeting defined soluble mediators, such as cytokines or their receptors, which are involved in the immune inflammatory pathways of the innate and adaptive immune responses.

Various autoimmune and inflammatory diseases are currently treated with FDA approved biologic agents that target cytokines: rheumatoid arthritis (Enbrel, Humira, Remicade, Simponi); Crohn's disease (Humira, Remicade); psoriasis (Enbrel, Humira, Remicade, Stelara), and age-related macular degeneration (Lucentis). In addition, various agents are in clinical development for other autoimmune and/or inflammatory diseases including Alzheimer's disease, asthma, COPD, graft-versus-host disease, pulmonary eosinophilia, multiple sclerosis, systemic lupus erythematosus, sepsis, and various forms of cancer.

While the advent of biologic agents that target cytokines has delivered high efficacy and good patient outcomes, there have also been negative effects from the use of these agents, including drug sensitization and the development of an immune response to the therapeutic agent. Other risks from such biologic agents include the possible reactivation of chronic viruses (e.g., hepatitis C, Epstein-Barr, varicella and JC virus). TNF-alpha blockers for example have been associated with reactivation of latent tuberculosis in treated patients resulting in much emphasis on the development of guidelines for use and for monitoring patients treated with anti-TNF biologic agents. Biologic agents have been shown to be associated with the development of serious, life-threatening infections in patients (e.g., Remicade, Enbrel, Humira, Actemra, Simponi & Stelara). Biologic agents are also connected with the appearance of specific cancers such as lymphoma, (Package Inserts for Remicade, Enbrel, Humira, Actemra, Simponi & Stelara). It has also been observed that the blockade of cytokines using many, if not all, of the biologic agents has led to the development of autoantibodies, lupus-like syndrome or glomerulonephritis. (Package Inserts for Remicade, Enbrel, Humira, Actemra, Simponi & Stelara).

The occurrence of such adverse events compromises the overall benefit-risk profile of the therapeutic agent. Moreover, the economic cost associated with the treatment of the sequelae that derive from the therapeutic agent decreases the cost-effectiveness of the agent relative to the treatment of the originally intended condition.

### Immunoapheresis System

The present immunoapheresis system addresses the deficiencies of currently available systemic treatments with the foregoing biologic agents, decreasing certain of the adverse events associated with these biologic agents while maintaining the intended benefits. This is accomplished by removing circulating cytokine and/or growth agent molecules from a subject and thereby reducing the unwanted effect of such molecules on the subject, without administering a biologic agent to the subject. The present therapy therefore specifically targets soluble cytokines and growth factors, rather than targeting cells or cell surface receptors.

The cytokine and/or growth agent molecules removed by the present system are generally removed via apheresis. Apheresis refers to the separation of blood into its individual constituents while returning the remainder of the blood to circulation. Depending on the substance to be removed, different processes are employed. For example, if separation by weight is required, different forms of centrifugation can be used. Alternatively, other methods of apheresis involve adsorption onto coated beads within a column. Plasmapheresis specifically refers to the collection of FFP (fresh frozen plasma), immune globulin products, platelets, leukocytes, red blood cells and/or other plasma derivatives. Extracorporeal cytokine apheresis has been employed in sepsis using CTR agarose beads as the therapeutic adsorbent to non-specifically remove cytokines and other molecules based upon their relatively small size. In contrast, the present system selectively removes cytokines from plasma using specific monoclonal antibodies or other specific binding agents for the protein ligand. Through immunoadsorption, the present system extends the practice of apheresis by selectively removing and/or reducing the levels of inflammatory cytokines and/or growth factors, thus reducing disease pathogenesis.

In one embodiment, the present system involves the blood of a patient being passed through a centrifugation apparatus that separates out the plasma, returning the non-required constituents back to circulation. An example of an apparatus that can be used in the present system is the AS104 cell plasma separator (Fresenius Hemocare, Redmond, Wash.). The centrifugation can be accomplished by methodologies that are in standard practice, such as continuous flow centrifugation (CFC). CFC generally requires two venipunctures so that blood can be collected, spun, and returned simultaneously, though newer systems use a single venipuncture. The main advantage of this system is the low extracorporeal volume (calculated by volume of the apheresis chamber, the donor's hematocrit, and total blood volume of the donor) used in the procedure, which can be advantageous in the elderly and for children.

Alternatively, intermittent flow centrifugation can be use. This technique works in cycles, taking blood, spinning/processing it and then giving back the necessary parts to the donor in a bolus. The main advantage is a single venipuncture site. To stop the blood from coagulating, anticoagulant is automatically mixed with the blood as it is pumped from the body into the apheresis machine.

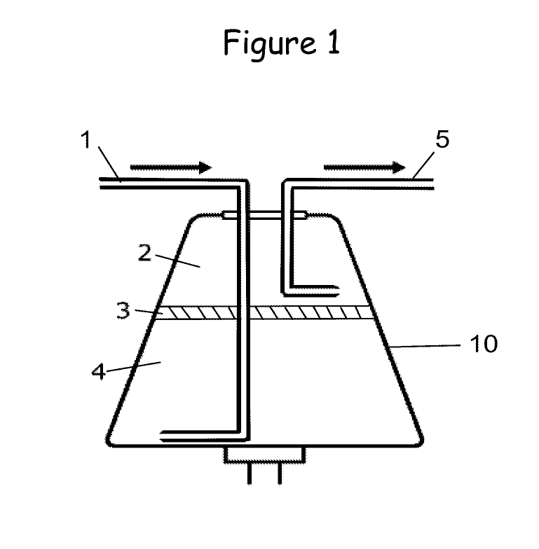

The centrifugation process in the present system is illustrated in FIG. 1. Whole blood enters the centrifuge 10 through a first conduit 1, and the centrifuge then separates the blood into plasma 2, leukocytes 3, and erythrocytes 4. Selected components, preferably plasma, are then drawn off through a second conduit 5 for further treatment with the present system.

Consistent with standard apheresis practice, a subject's fluid generally should be replaced to keep correct intravascular volume. Preferably, the system removes only relatively small amounts of fluid (not more than 10.5 mL/kg body weight). If a crystalloid like normal saline is used, the infusion amount should be triple what is removed, as the three to one ratio of normal saline for plasma is needed to keep up oncotic pressure. Other replacement fluids include normal serum albumin and fresh frozen plasma. The total plasma exchange preferably occurs at between 100 and 150 milliliters per hour, until between about 1 and 1.5 plasma volumes have been exchanged.

Vascular access can be attained via antecubital veins or through an internal jugular double lumen catheter, for example. Computer-controlled elution monitors (e.g., a Citem 10 monitor) are set up to pump plasma between the immunoadsoprtion columns to which are affixed the selected monoclonal antibodies or other specific binding partners targeting specific cytokines or growth factors.

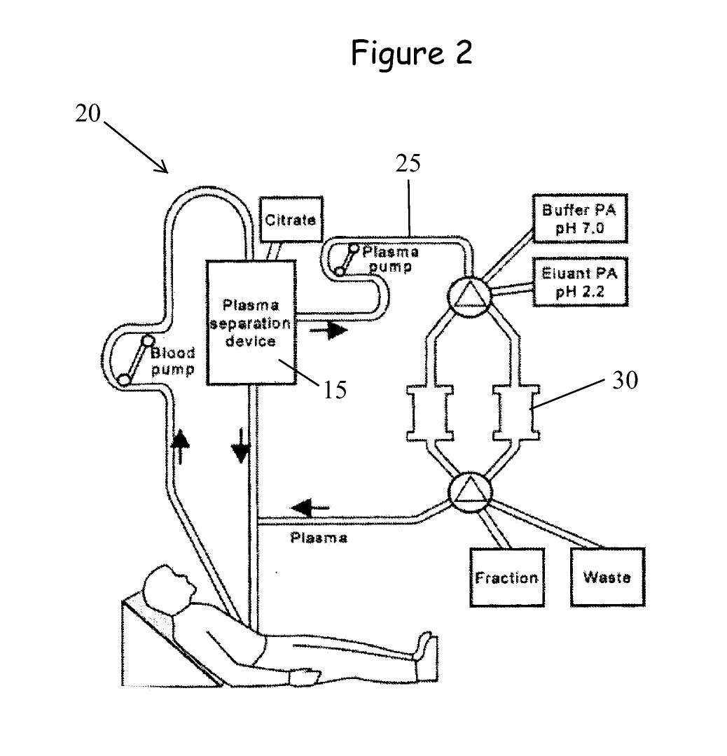

FIG. 2 illustrates an embodiment of the present system 20. In an adsorption cycle of the system 20, plasma is conducted from a plasma separation device 15 to buffer-washed columns 30 to allow cytokines and/or growth factors to bind to monoclonal antibodies covalently bound to the immunoabsorbant columns 30. In this illustrated embodiment, the vascular access conduit 25 leading to the columns 30 is in communication with the subject's vasculature through an indirect connection, i.e. via the plasma separation device 15. The columns can comprise a substrate such as Sepharose 4B, agarose, protein-A silica gel, or Staphylococcal protein A-agarose (SPA), for example, to which the monoclonal antibodies or other specific binding partners of the present system are bound. Specific columns that can be used in the present system include columns marketed as Ig-Therasorb® (Therasorb, Baxter Healthcare Corporation, Deerfield, Ill.), Prosorba (Cypress Bioscience, Inc.), Hemocleanse-PF (Hemocleanse), and Selesorb (Kaneka, Osaka, Japan).

Any of a number of specific binding agents which specifically bind a cytokine or growth factor can be retained by the substrate. In one embodiment, monoclonal antibodies such as ACTEMRA (tocilizumab), ENBREL (etanercept), HUMIRA (adalimumab), REMICADE (infliximab), SIMPONI (golimumab) and/or STELARA (ustekinumab) can be bound to the substrate. Other specific binding partners known to the art can be used, or alternatively can be identified using a variety of selection technologies which are known for the identification and isolation of proteins with certain binding characteristics and affinities. These include, for example, display technologies such as phage display, ribosome display, cell surface display, and the like. Methods for production and screening of antibody variants are also well known in the art. General methods for antibody molecular biology, expression, purification, and screening are described in Antibody Engineering, Duebel & Kontermann eds., Springer-Verlag, Heidelberg (2001); Hayhurst & Georgiou, 2001, Curr Opin Chem Biol, 5:683-689; and Maynard & Georgiou, 2000, Annu Rev Biomed Eng, 2:339-76.

Reusable columns containing single or multiple antibodies [e.g., ACTEMRA (tocilizumab), ENBREL (etanercept), HUMIRA (adalimumab), REMICADE (infliximab), SIMPONI (golimumab) and/or STELARA (ustekinumab)] as well as others that target soluble cytokines, chemokines and other growth factors and/or modulators can be used in the present system. The columns can be assembled by covalently coupling the monoclonal antibodies or other specific binding partners of choice to standard Prosorba or Sepharose CL-4B type columns containing protein A, for example. The binding partners can be bound to the substrate of the column in ways known to the art.

Upon saturation of the columns or before, the treated plasma is recombined with cellular components (if these have not already been returned to the subject's vasculature) and returned to the patient. Saturated columns are preferably washed and regenerated, after which an elution monitor can switch the plasma flow over a second column to allow immunoadsorption to continue. The daily treatment time can range from 3 to 5 hours, during which an average of between one and two patient blood volumes can be processed, e.g. through 20-30 adsorption cycles per column. Following treatment the columns can be washed and preserved with thimerosol and can be used for the same patient on subsequent treatments, if desired. The columns are preferably stored at 2-8° C. until the next treatment.

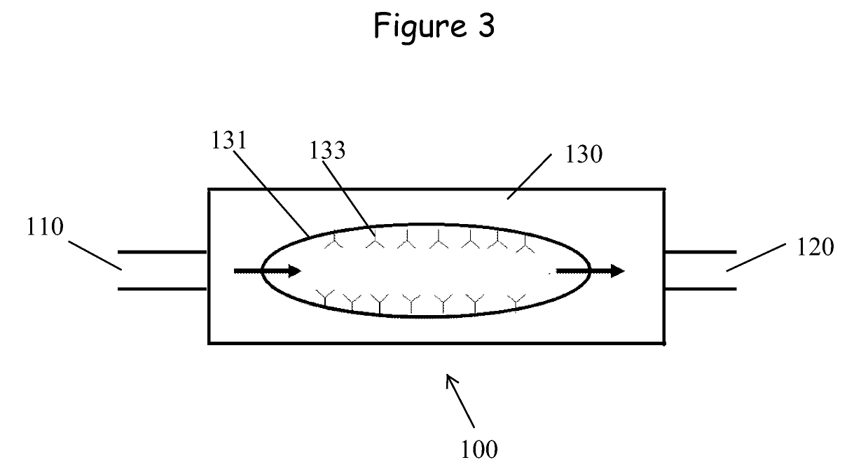

In a second embodiment, illustrated in FIG. 3, the present method involves the blood of a patient being passed through a device which is worn by the patient throughout the treatment period, either extracorporeally or as an implant. The device 100 in this embodiment comprises an inlet conduit 110, an outlet conduit 120, and a cartridge 130 comprising an inner substrate or cassette 131 on which are bound therapeutic antibodies or other binding partners 133. The substrate 131 can be, for example, a semi-permeable polymeric or biopolymeric membrane which excludes cells and cellular components and allows primarily blood plasma to pass through. The antibodies 133 on the interior of the substrate can be bound to it using means known to the art, such as an avidin-biotin binding system.

Blood or other body fluid of a subject can be directed through the cassette 131 of the device 100 using a blood pump. A number of blood pumps are known to the art, including pumps designed to be implanted such as the pump disclosed in U. S. Pat. No. 6,641,612. The pump can be placed in-line with the flow of blood or other fluid through the device 100, either upstream or downstream from the cassette 131.

The device 100 preferably separates cellular blood cells and other components from plasma entering the cassette 131 in order to maximize contact between the antibodies or other specific binding partners 133. In one embodiment, such components are prevented from entering the cassette through size exclusion. In this embodiment, the cassette 131 can be formed from a membrane which retains the specific binding partners 133 on an interior surface or compartment of the membrane. The membrane is preferably formed from a biocompatible polymer or other material which forms a mesh or otherwise comprises pores of less than 7 microns in size, and more preferably less than 2 or 3 microns in size. The device 100 may alternatively or in addition contain various polymers or biopolymers to enact such filtration. In another embodiment, the inner cassette 131 removes cytokines through absorption of plasma constituents onto beads coated with an absorbent material (SPA) to which has been bound one or more of the specific monoclonal antibodies.

A determination of the efficacy of the present treatment in human subjects can be evaluated first in animal models. Experimentally induced polyarthritis can be induced in rats and mice, with the most frequently used models being adjuvant-induced arthritis (AA) in rats and type II collagen-induced arthritis (CIA) in mice or rats. Experimental autoimmune encephalomyelitis (EAE) rat models retain some qualities of the human syndrome of multiple sclerosis (MS). These models have been used to assess the efficacy of novel chemotherapies and the therapeutic effects of monoclonal antibodies against targets of interest (e.g. cytokines). They can also be utilized to evaluate extracorporeal immunoadsorption treatment to enhance the benefit:risk profile for selected biological therapies. Such experimentation can proceed as summarized in the examples provided by Kalden in his analysis of the biologic agents in the therapy of inflammatory rheumatic diseases, including therapeutic antibodies, cytokines, and cytokine antagonists.

The treatment frequency can be determined through preclinical and/or clinical experimentation, and can be customized on a per patient basis dependent upon the type (e.g., skin versus neurological) and severity of the diseases in question. For chronic use, treatments preferably occur no more frequently than once a week, and more preferably only every few weeks or months. The methodology described herein therefore is indicated for any patient with any inflammatory disease that is both severe and refractory to standard of care treatments.

In one example, a monoclonal antibody targeting a tumor necrosis factor, such as the molecule available as ENBREL, is bound to a PROSORBA column. Enbrel is a dimeric fusion protein consisting of the extracellular ligand-binding portion of the human 75 kilodalton (p75) tumor necrosis factor receptor (TNFR) linked to the Fc portion of human IgG1. It is this Fc portion that is contained on all monoclonal antibodies and many fusion proteins that will bind to the protein A contained in either the PROSORBA or Sephraose CL-4B columns. The column is provided in a device as shown in FIG. 2, and a subject's vasculature is placed in fluid communication with the column. The subject's plasma is fractionated from blood and passed through the PROSORBA column until between 1 to 1.5 plasma volumes are exchanged, and the treated plasma is then returned to the subject. The foregoing regimen can be applied to subjects diagnosed with rheumatoid arthritis, polyarticular juvenile idiopathic arthritis, psoriatic arthritis, ankylosing spondylitis and chronic plaque psoriasis.

Although the present invention has been described in considerable detail with reference to certain preferred embodiments, other embodiments are possible. The steps disclosed for the present methods, for example, are not intended to be limiting nor are they intended to indicate that each step is necessarily essential to the method, but instead are exemplary steps only. Therefore, the scope of the appended claims should not be limited to the description of preferred embodiments contained in this disclosure.

Recitation of value ranges herein is merely intended to serve as a shorthand method for referring individually to each separate value falling within the range. Unless otherwise indicated herein, each individual value is incorporated into the specification as if it were individually recited herein. All references cited herein are incorporated by reference in their entirety.

### REFERENCES

1. Gilman A, Goodman L S, Hardman J G, Limbird L E (2001). Goodman & Gilman's the pharmacological basis of therapeutics. New York: McGraw-Hill. ISBN 0-07-135469-7.
2. Cannon J G (2000). “Inflammatory Cytokines in Nonpathological States”. News Physiol Sci. 15: 298-303. PMID 11390930.
3. Erickson, A R, Mikuls, T R, Switching anti-TNF agents: what is the evidence? 2007, Current Rheumatologic Rep. 9 (5): 416-420.
4. Vassilopoulos, D, Calabrese, L H, 2007. Risks of immunosuppressive therapies including biologic agents in patients with rheumatic diseases and co-existing chronic viral infections. Curr. Opin. Rheumatol. 19 (6): 619-625.
5. Vielhauer, V., Mayadas, T. N., 2007. Functions of TNF and its receptors in renal disease: distinct roles in inflammatory tissue injury and immune regulation. Semin. Nephrol. 27 (3): 286-308.
6. Fuchs-Polychuck, I, Avnon, L S, Abu-Shakra, M. 2007. Tuberculosis among patients treated with TNF-alpha blockers. Harefuah 146(3):212-8.
7. Saketkoo, L A, Espinoza, L. R. 2006. Impact of biologic agents on infectious diseases. Infect. Dis. Clin. North. Am. 20 (4): 931-61.
8. Barclay, L. 2005. IgG immunoadsorption may improve global disease activity in systemic lupus erythematosus. Annals of Rheumatic Disease, July 2005.
9. Jovin, I S, Taborski, U., Muller-Berghaus, G. 2000. Analysis of the long term efficacy and selectivity of immunoadsorption columns for low density lipoprotein apheresis. ASAIO J. 46 (3): 298-300.
10. Shimizu, T, Endo, Y. Tani, T 2006. Extracorporeal cytokine apheresis for sepsis: CTR as a new therapeutic adsorbent Critical Care Medicine—Volume 34, Issue 3 (March 2006).
11. A. Du Moulin, J. Müller-Derlich, F. Bieber, W. O. Richter, U. Frei, R. Müller, R. Spaethe. 1993. Antibody-Based Immunoadsorption as a Therapeutic Means. Blood Purif. 11: 145-149.
12. Dommasch, E. & J. M. Gelfand. 2009. Is there truly a risk of lymphoma from biologic therapies? Dermatol Ther. 22(5): 418-30.
13. Patel, R V, Clark, L N, Lebwohl, M. & J M Weinberg. 2009. Treatments for psoriasis and the risk of malignancy. J. Am. Acad. Dermatol. 60(6): 1001-17.
14. Setoguci, S., Solomon, D H, Weinblatt, M. E., Katz, J N, Avron, J., Glynn, R J, Cook, E. F., Carney, G. & S. Schneeweiss. 2006. Tumor necrosis factor alpha antagonist use and cancer in patients with rheumatoid arthritis. Arthritis Rheum 54(9): 2757-64.
15. Curtis, J R, Patkar, N., Xie, A., Martin, C., Allison, J J, Saag, M., Shatin, D. & K G Saag. 2007. Risk of serious bacterial infections among rheumatoid arthritis patients exposed to tumor necrosis factor alpha antagonists. Nat Clin Pract Rheumatol 3(10):546-7.
16. Chen Y F, Jobanputra, P., Barton, P, Jowett, S., Bryan, S., Clark, W., Fry-Smith, A., & A. Burls. 2006. A systematic review of the effectiveness of adalimumab, etanercept and infliximab for the treatment of rheumatoid arthritis in adults and an economic evaluation of their cost-effectiveness. Heath Technol. Assess. 10 (42): 1-229.
17. Cooper, L. T., Belohlavek, M., Korinek, J., Yoshifuku, S., Senupta, P. P., Burgstaler, E. A., and J. L. Winters. 2007. A Pilot Study to Assess the Use of Protein A Immunoadsorption for Chronic Dilated Cardiomyopathy. J. Clin. Apheresis. 22: 210-214.
18. Yoshino S, Cleland L G. 1992. Depletion of alpha/beta T cells by a monoclonal antibody against the alpha/beta T cell receptor suppresses established adjuvant arthritis, but not established collagen-induced arthritis in rats. J Exp Med 175:907-15.
19. Kalden J R. 1994. Biologic agents in the therapy of inflammatory rheumatic diseases, including therapeutic antibodies, cytokines, and cytokine antagonists. Curr Opin Rheumatol 6:281-6.

1. A system for treating an autoimmune or inflammatory disease of a subject, comprising:
a vascular access conduit adapted at a first end for removing a body fluid from the subject, the body fluid comprising blood or plasma and a cytokine or a growth factor;
a chromatography system comprising a solid substrate and having an inlet end and an outlet end, the substrate having one or more specific binding partners for a cytokine or a growth factor bound thereto, wherein the inlet end of the chromatography system is in fluid communication with a second end of the vascular access conduit;
a fluid return conduit having a first end in fluid communication with the outlet end of the chromatography system and a second end in fluid communication with the vasculature of the subject.

2. The system of claim 1, wherein the specific binding partners are antibodies.

3. The system of claim 2, wherein the antibodies are monoclonal antibodies selected from the group consisting of tocilizumab, etanercept, adalimumab, infliximab, golimumab and ustekinumab.

4. The system of claim 2, wherein the antibodies are immobilized in a polymer matrix that excludes blood cells.

5. The system of claim 1, wherein the cytokine or growth factor is selected from the group consisting of an interferon, a tumor necrosis factor (TNF), an interleukin (IL), and a chemokine.

6. The system of claim 1, wherein the cytokine or growth factor is selected from the group consisting of interferon alpha, interferon beta, interferon gamma, TNF-alpha, TNF-beta, IL-1, IL-2, IL-3, IL-4, IL-5, IL-6, IL-7, IL-8, IL-10, IL-12, IL-13, IL-15, IL-17, IL-18, IL-20, IL-22, IL-23, MCP-1, MT, RANTES, and MIP-1-alpha.

7. The system of claim 1, wherein the cytokine or growth factor are selected from the group consisting of VEGF, FGF, EGF, and PDGF.

8. The system of claim 1, wherein the chromatography system comprises a column, and wherein the solid substrate is selected from the group consisting of sepharose, agarose, and silica gel.

9. The system of claim 1, wherein the system comprises an apheresis unit for separating blood plasma from the subject's blood.

10. The system of claim 1, wherein the apheresis unit comprises a centrifuge.

11-13. (canceled)

14. A method for treating an autoimmune or inflammatory disease of a subject, comprising the steps of:
removing a body fluid from the subject, wherein the body fluid comprises blood or plasma and a cytokine or a growth factor;
conducting the body fluid or a fraction thereof to a substrate, the substrate having one or more specific binding partners for the cytokine or the growth factor bound thereto, wherein the body fluid or fraction thereof is placed in contact with the specific binding partners;
returning the body fluid or fraction thereof to the patient after it is placed in contact with the specific binding partners.

15. The method of claim 14, wherein the disease being treated is selected from the group consisting of rheumatoid arthritis, Crohn's disease, psoriasis, age-related macular degeneration, Alzheimer's disease, asthma, COPD, graft-versus-host disease, pulmonary eosinophilia, multiple sclerosis, systemic lupus erythematosus, sepsis, malignancies and cancer.

16. The use of claim 14, wherein a fraction of the body fluid is conducted through a solid substrate, and wherein the fraction comprises blood plasma.
